(19) United States
(12) Patent Application Publication
(10) Pub. No.: US 2005/0059088 A1
(43) Pub. Date: Mar. 17, 2005

(54) Alternatively spliced isoforms of receptor-interacting serine-threonine kinase 2 (RIPK2)

(76) Inventors: Christopher D. Armour, Kirkland, WA (US); John C. Castle, Seattle, WA (US); Philip W. Garrett-Engele, Seattle, WA (US); Zhengyan Kan, Bellevue, WA (US); Patrick M. Loerch, Boston, MA (US); Nicholas F. Tsinoremas, Sammamish, WA (US)

Correspondence Address: R. Douglas Bradley;Merck & Co., Inc., Patent Department RY60-30, P.O. Box 2000, Rahway, NJ, 07065-0907, US

(21) Appl. No.: 10/901,779

(22) Filed: Jul. 29, 2004

Related U.S. Application Data
(60) Provisional application No. 60/492,038, filed on Aug. 1, 2003.

(51) Int. Cl. G01N 33/53; C07H 21/04; C07K 14/705

(52) U.S. Cl. 435/7.1; 530/350; 435/69.1; 435/320.1; 435/325; 536/23.5

(57) ABSTRACT

The present invention features nucleic acids and polypeptides encoding three novel splice variant isoforms of receptor-interacting serine-threonine kinase 2 (RIPK2). The polynucleotide sequences of RIPK2sv1.1, RIPK2sv1.2, and RIPK2s2 are provided by SEQ ID NO 1, SEQ ID NO 3, and SEQ ID NO 5, respectively. The amino acid sequences for RIPK2sv1.1, RIPK2sv1.2, and RIPK2sv2 are provided by SEQ ID NO 2, SEQ ID NO 4, and SEQ ID NO 6, respectively. The present invention also provides methods for using RIPK2sv1.1, RIPK2sv1.2, and RIPK2sv2 polynucleotides and proteins to screen for compounds that bind to RIPK2sv1.1, RIPK2sv1.2, and RIPK2sv2, respectively.

This application claims priority to U.S. Provisional Patent Application Ser. No. 60/492,038 filed on Aug. 1, 2003, which is incorporated by reference herein in its entirety.

### BACKGROUND OF THE INVENTION

The references cited herein are not admitted to be prior art to the claimed invention.

RIPK2 (also called RICK, RIP2, CARDIAK, and CARD3) has been implicated in a variety of functions including: integrating signals for innate and adaptive immune systems, regulating apoptosis, controlling a myogenic differentiation checkpoint, and regulating nuclear-factor-kappa-beta (NFkB) and Jun N-terminal kinase (JNK) activation. RIPK2 contains an N-terminal serine/threonine kinase catalytic domain (amino acids 1-294) and a C-terminal caspase activation and recruitment domain (CARD; amino acids 430-540) (Inohara et. al., 1998, J Biol. Chem. 273: 12296-12300; Thome et. al., 1998, Curr Biol. 8: 885-888; McCarthy et. al., 1998, J Biol. Chem. 273: 16968-16975). CARDs mediate homophilic protein interactions that allow for the recruitment of caspases to receptor complexes and have been identified in a number of proteins involved in apoptotic signaling (Hofmann et. al., 1997, Trends Biochem. Sci. 22: 155-156). The N-terminal kinase domain has been shown to autophosphorylate RIPK2 in vitro (Inohara et. al., 1998; Thome et. al., 1998; McCarthy et. al., 1998).

Transcript expression analysis indicates that RIPK2 mRNA is expressed in several human tissues including heart, brain, placenta, testis, lung, pancreas, spleen, lymph node, and peripheral blood lymphocytes. (Inohara et. al., 1998; Thome et. al., 1998; McCarthy et. al., 1998). Two RIPK2 transcripts of 1.8 kilobases (kb) and 2.5 kb have been reported (Inohara et. al., 1998; Thome et. al., 1998; McCarthy et. al., 1998). The difference in transcript length is the result of alternative polyadenylation (Inohara et. al., 1998).

The effect of overexpression of RIPK2 on apoptosis varies depending on the cell type in which RIPK2 is expressed. Expression of RIPK2 in 293T cells does not induce apoptosis by itself (Inohara et. al., 1998; Thome et. al., 1998), but does augment apoptosis induced by Caspase-8 in a kinase and CARD domain dependent manner (Inohara et. al., 1998). Expression of RIPK2 in MCF7 breast carcinoma cells, in contrast, induced apoptosis by itself (McCarthy et. al., 1998). Induction of apoptosis in MCF7 cells was found to be dependent on the presence of the RIPK2 CARD domain but not on RIPK2 kinase activity (McCarthy et. al., 1998). In Schwann cells, RIPK2 expression abrogated apoptosis stimulated by activation of a p75 receptor (Khursigara et. al., 2001. J. Neurosci. 21: 5854-5863). Thus, expression of RIPK2 has been reported to both induce and abrogate apoptosis depending on the cell type used.

Apoptosis, or programmed cell death, is an integral part of development and removes surplus or harmful cells from the body (reviewed in Nagata, S., 1997, Cell 88: 355-365). However, defective regulation of apoptosis is implicated in the development of several disease states as well as ageing. For example, excessive apoptosis is associated with neurological disorders (such as Alzheimer's, Parkinson's and Huntington's disease) and AIDS, while deficient apoptosis is associated with cancer, auto-immunity, and viral infections (reviewed in Friedlander, R. M., 2003, N Engl. J Med. 348: 1365-75). Because expression of RIPK2 affects the regulation of apoptosis in a variety of cell types, RIPK2 activity may be an important factor in the development of disease states in which regulation of apoptosis is critical. Significantly, RIPK2 protein level is increased in the frontal cortex of patients with Alzheimer's disease (Engidawork et. al., 2001, Biochem. Biophys. Res. Commun. 281: 84-93).

Overexpression of RIPK2 activates NFkB in 293T cells (Thome et. al., 1998; McCarthy et. al., 1998; Medzhitov et. al., 2000, Immunol. Rev. 173: 89-97), HEK 293 cells (Khursigara et. al., 2001), and wild type MEF's (Inohara et. al., 2000, J. Biol. Chem. 275: 27823-27831). Although expression of the CARD domain of RIPK2 weakly induced NFkB (Thome et. al., 1998), full activation of NFkB required full length RIPK2 protein (Thome et. al., 1998; McCarthy et. al., 1998; Medzhitov et. al., 2000). However, a mutation in the kinase catalytic domain of RIPK2 did not fully abrogate the ability of RIPK2 to activate NFkB (Thome et. al., 1998; McCarthy et. al., 1998; Inohara et. al., 2000). Although the mechanism of NFKB activation by RIPK2 is not known, RIPK2 has been shown to bind to IKK-γ, a regulatory subunit of the IKK complex which is essential for induction of NFkB activation by tumor necrosis factor alpha (TNF-α) and interleukin-1 (IL-1) (Inohara et. al., 2000). Thus, RIPK2 could modulate NFkB activity through its interaction with IKK-γ.

NFkB is a transcription factor that plays an integral role in the cellular response to a wide array of stimuli, including cytokines, such as TNF-α and IL-1, bacterial lipopolysaccharide (LPS), viral infection, phorbol esters, UV radiation, and free radicals. NFkB regulates genes involved in immune function, inflammation responses, growth control, cell death, cell adhesion, and viral replication (for reviews see Baldwin, A. S., 1996, Annu. Rev. Immunol. 14, 649-681; Baeuerle, P. A. & Baltimore, D., 1996, Cell 87, 13-20; Stancovski, I. & Baltimore, D., 1997 Cell, 91, 299-302). The function of NFkB has been implicated in diseases as varied as rheumatoid arthritis, lupus, HIV-AIDS, influenza, septic shock, atherosclerosis, oncogenesis, and apoptosis (Baldwin, 1996). Regulation of NFkB by RIPK2 is therefore likely to have a significant impact on cellular processes and disease onset and progression.

RIPK2 kinase activity directly activates ERK1 and ERK2 (Navas et. al., 1999, J. Biol. Chem. 274: 33684-33690). The ERK/MAPK signaling pathway is critical for a number of biological processes including proliferation and differentiation (Lewis et. al., 1998, Adv. Cancer Res. 74: 49-139; Robinson et. al., 1997, Curr. Opin. Cell Biol. 9: 180-186). RIPK2 kinase-deficient mutants block the activation of ERK2 by TNF-α and physically interact with components of the ERK signaling pathway including Raf1 (Navas et. al., 1999). The role of RIPK2 in the ERK signaling pathway may explain the requirement for RIPK2 in the regulation of myogenic differentiation. Overexpression of RIPK2 prevents cultured myoblasts from differentiating, resulting in continued proliferation (Munz et. al., 2002, Mol. Cell. Biol. 22: 5879-5886). While intact RIPK2 protein is required to prevent differentiation, mutation of the kinase catalytic domain does not abrogate inhibition of myogenic differentiation, indicating that the kinase activity of RIPK2 is not required for the checkpoint function of RIPK2 in myogenic differentiation (Munz et. al., 2002).

Analysis of RIPK2 deficient mice indicates that RIPK2 is required for regulation of innate and adaptive immune and inflammatory responses. RIPK2 deficient mice were born in the expected Mendelian ratio, and showed no gross developmental abnormalities or abnormal composition of lymphocytes as determined by flow cytometry (Kobayashi et. al., 2002, Nature 416: 194-199; Chin et. al., 2002, Nature 416: 190-194). However, these mice exhibited a decreased ability to defend against infection by the intracellular pathogen Listeria monocytogenes (Chin et. al., 2002). RIPK2 deficient macrophages and T-cells showed severely reduced NFkB activation (Kobayashi et. al., 2002; Chin et. al., 2002). RIPK2 deficiency also resulted in impaired interferon-γ production in both T_{H}1 and natural killer cells and impaired T_{H}1-cell differentiation (Kobayashi et. al., 2002; Chin et. al., 2002). Analysis of RIPK2 deficient mice suggests that RIPK2 is a candidate target for immune intervention.

RIPK2 has been reported to physically associate with several proteins involved in receptor mediated signaling through the tumor necrosis factor (TNF) family of receptors including TNFR-1, TNFR-2, Fas (CD-95/APO-1), lyphotoxin-β receptor, CD40, CD30, OX-40, DR3, DR4, and DR5. For example, RIPK2 physically interacts with CLARP, a caspase-related protein that interacts with Caspase-8 and FADD (a protein which associates with the Fas/CD-95 and TNFR-1 receptors) (Inohara et. al., 1998). CLARP could therefore function as an adapter molecule to link RIPK2 to proximal components of the receptor signaling complex.

RIPK2 also physically interacts with Caspase-1 (Thome et. al., 1998; Humke et. al, 2000, Cell 103: 99-111). This protein interaction is mediated by CARD domains in the C-terminus of RIPK2 and in the prodomain of Caspase-1 (Thome et. al., 1998; Humke et. al., 2000). RIPK2 enhances the activation of Caspase-1 by promoting its oligomerization which leads to processing of adjacent pro-Caspase-1 protein (Humke et. al., 2000). The association between RIPK2 and Caspase-1 can be abrogated by the ICEBERG protein, which inhibits and/or displaces RIPK2 by binding Caspase-1 through its own CARD domain. (Humke et. al., 2000).

RIPK2 has been reported to associate directly with p75 receptor in a nerve growth factor (NGF) dependent fashion (Khursigara et. al., 2001) and with several receptor associating proteins including TRAF1, TRAF2, TRAF5, and TRAF6 (Thome et. al., 1998; McCarthy et. al., 1998). Co-expression of CD40 receptor, RIPK2, TRAF1 and TRAF2 resulted in association of RIPK2 with CD40 (McCarthy et. al., 1998). Likewise, co-expression of TNFR-1 receptor, RIPK2, TRADD, TRAF1 and TRAF2 resulted in association of RIPK2 with TNFR-1 (McCarthy et. al., 1998). Collectively, these data suggest that RIPK2 is a component of the p75, CD40, Fas/CD-95 and TNFR-1 receptor signaling complexes.

RIPK2 activity appears to be altered by interaction with ligands. For example, expression of polypeptides comprising CARD domains with high affinity for RIPK2 protein binding partners may prevent RIPK2 from physically associating with other CARD domain containing proteins (Humke et. al., 2000). Protein-protein interactions mediated by CARD domains have also been reported to be disrupted by nitric oxide (NO) (Zech et. al., 2003, Biochem J. 371(Part 3): 1055-64). Compounds that alter the serine-kinase activity of RIPK2 may also influence RIPK2 function. Methods for assessing the kinase activity of RIPK2 have been described (Inohara et. al., 1998; Thome et. al., 1998; McCarthy et. al., 1998; Navas et. al., 1999). Methods for screening for compounds that modulate serine-threonine kinase activity have been disclosed (US2003/0134310A1; WO 02/14542). In addition, anti-sense oligonucleotides designed to inhibit RIPK2 have been described (U.S. Pat. No. 6,426,221 B1).

Because of the multiple therapeutic values of compounds targeting receptor mediated signaling pathways that modulate apoptosis, regulation of NFkB, cellular differentiation, and immune response, and the essential regulatory role played by RIPK2, there is a need in the art for compounds that selectively bind to isoforms of RIPK2. The present invention is directed towards two novel RIPK2 isoforms (RIPK2sv1 and RIPK2sv2) and uses thereof.

### BRIEF DESCRIPTION OF THE FIGURES

FIG. 1A illustrates the exon structure of RIPK2 mRNA corresponding to the known long reference form of RIPK2 mRNA (labeled NM_{—}003821.2) and the exon structure corresponding to the inventive short form splice variants (labeled RIPK2sv1 and RIPK2sv2). FIG. 1B depicts the nucleotide sequences of the exon junctions resulting from the splicing of exon 1 to exon 3 in the case of RIPK2sv1 mRNA and the splicing of exon 7 to exon 9 in the case of RIPK2sv2 mRNA. In FIG. 1B, in the case of RIPK2sv1, the nucleotides shown in italics represent the 20 nucleotides at the 3′ end of exon 1 and the nucleotides shown in underline represent the 20 nucleotides at the 5′ end of exon 3; and in the case of RIPK2sv2, the nucleotides shown in italics represent the 20 nucleotides at the 3′ end of exon 7 and the nucleotides shown in underline represent the 20 nucleotides at the 5′0 end of exon 9.

### SUMMARY OF THE INVENTION

Microarray experiments and RT-PCR have been used to identify and confirm the presence of novel splice variants of human RIPK2 mRNA. More specifically, the present invention features polynucleotides encoding different protein isoforms of RIPK2. A polynucleotide sequence encoding RIPK2sv1.1 is provided by SEQ ID NO 1. An amino acid sequence for RIPK2sv1.1 is provided by SEQ ID NO 2. A polynucleotide sequence encoding RIPK2sv1.2 is provided by SEQ ID NO 3. An amino acid sequence for RIPK2sv1.2 is provided by SEQ ID NO 4. A polynucleotide sequence encoding RIPK2sv2 is provided by SEQ ID NO 5. An amino acid sequence for RIPK2sv2 is provided by SEQ ID NO 6.

Thus, a first aspect of the present invention describes a purified RIPK2sv1.1 encoding nucleic acid, a purified RIPK2sv1.2 encoding nucleic acid, and a purified RIPK2sv2 encoding nucleic acid. The RIPK2sv1.1 encoding nucleic acid comprises SEQ ID NO 1 or the complement thereof. The RIPK2sv1.2 encoding nucleic acid comprises SEQ ID NO 3 or the complement thereof. The RIPK2sv2 encoding nucleic acid comprises SEQ ID NO 5 or the complement thereof. Reference to the presence of one region does not indicate that another region is not present. For example, in different embodiments the inventive nucleic acid can comprise, consist, or consist essentially of an encoding nucleic acid sequence of SEQ ID NO 1, can comprise, consist, or consist essentially of the nucleic acid sequence of SEQ ID NO 3, or alternatively can comprise, consist, or consist essentially of the nucleic acid sequence of SEQ ID NO 5.

Another aspect of the present invention describes a purified RIPK2sv1.1 polypeptide that can comprise, consist or consist essentially of the amino acid sequence of SEQ ID NO 2. An additional aspect describes a purified RIPK2sv1.2 polypeptide that can comprise, consist, or consist essentially of the amino acid sequence of SEQ ID NO 4. An additional aspect describes a purified RIPK2sv2 polypeptide that can comprise, consist, or consist essentially of the amino acid sequence of SEQ ID NO 6.

Another aspect of the present invention describes expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 4, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 6, wherein the nucleotide sequence is transcriptionally coupled to an exogenous promoter.

Alternatively, the nucleotide sequence comprises, consists, or consists essentially of SEQ ID NO 1, and is transcriptionally coupled to an exogenous promoter. In another embodiment, the nucleotide sequence comprises, consists, or consists essentially of SEQ ID NO 3, and is transcriptionally coupled to an exogenous promoter. In another embodiment, the nucleotide sequence comprises, consists, or consists essentially of SEQ ID NO 5, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention, describes a recombinant cell made by a process comprising the step of introducing into the cell an expression vector comprising a nucleotide sequence comprising, consisting, or consisting essentially of SEQ ID NO 1, SEQ ID NO 3, or SEQ ID NO 5, or a nucleotide sequence encoding a polypeptide comprising, consisting, or consisting essentially of an amino acid sequence of SEQ ID NO 2, SEQ ID NO 4, or SEQ ID NO 6, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing RIPK2sv1.1, RIPK2sv1.2, or RIPK2sv2 polypeptide comprising SEQ ID NO 2, SEQ ID NO 4, or SEQ ID NO 6, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to RIPK2sv1.1 as compared to one or more RIPK2 isoform polypeptides that are not RIPK2sv1.1. In another embodiment, a purified antibody preparation is provided comprising antibody that binds selectively to RIPK2sv2 as compared to one or more RIPK2 isoform polypeptides that are not RIPK2sv2.

Another aspect of the present invention provides a method of screening for a compound that binds to RIPK2sv1.1, RIPK2sv2 or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a labeled RIPK2 ligand that binds to said polypeptide and a test preparation comprising one or more test compounds; (c) and measuring the effect of said test preparation on binding of said labeled RIPK2 ligand to said polypeptide comprising SEQ ID NO 2. Alternatively, this method could be performed using SEQ ID NO 6 instead of SEQ ID NO 2.

In another embodiment of the method, a compound is identified that binds selectively to RIPK2sv1.1 polypeptide as compared to one or more RIPK2 isoform polypeptides that are not RIPK2sv1.1. This method comprises the steps of: providing a RIPK2sv1.1 polypeptide comprising SEQ ID NO 2; providing a RIPK2 isoform polypeptide that is not RIPK2sv1.1, contacting said RIPK2sv1.1 polypeptide and said RIPK2 isoform polypeptide that is not RIPK2sv1.1 with a test preparation comprising one or more test compounds; and determining the binding of said test preparation to said RIPK2sv1.1 polypeptide and to RIPK2 isoform polypeptide that is not RIPK2sv1.1, wherein a test preparation that binds to said RIPK2sv1.1 polypeptide but does not bind to said RIPK2 isoform polypeptide that is not RIPK2sv1.1 contains a compound that selectively binds said RIPK2sv1.1 polypeptide. Alternatively, the same method can be performed using RIPK2sv2 polypeptide comprising, consisting, or consisting essentially of SEQ ID NO 6.

In another embodiment of the invention, a method is provided for screening for a compound able to bind to or interact with a RIPK2sv1.1 protein or a fragment thereof comprising the steps of: expressing a RIPK2sv1.1 polypeptide comprising SEQ ID NO 2 or a fragment thereof from a recombinant nucleic acid; providing to said polypeptide a labeled RIPK2 ligand that binds to said polypeptide and a test preparation comprising one or more compounds; and measuring the effect of said test preparation on binding of said labeled RIPK2 ligand to said polypeptide, wherein a test preparation that alters the binding of said labeled RIPK2 ligand to said polypeptide contains a compound that binds to or interacts with said polypeptide. In an alternative embodiment, the method is performed using RIPK2sv2 polypeptide comprising, consisting, or consisting essentially of SEQ ID NO 6 or a fragment thereof.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “RIPK2” refers to a receptor-interacting serine-threonine kinase 2 (NP_{—}003812). In contrast, reference to an RIPK2 isoform includes NP_{—}003812 and other polypeptide isoform variants of RIPK2.

As used herein, “RIPK2sv1.1”, “RIPK2sv1.2”, and “RIPK2sv2” refer to splice variant isoforms of human RIPK2 protein, wherein the splice variants have the amino acid sequence set forth in SEQ ID NO 2 (for RIPK2sv1.1), SEQ ID NO 4 (for RIPK2sv1.2), and SEQ ID NO 6 (for RIPK2sv2).

As used herein, “RIPK2” refers to polynucleotides encoding RIPK2.

As used herein, “RIPK2sv1” refers to polynucleotides that are identical to RIPK2 encoding polynucleotides, except that the sequence represented by exon 2 of the RIPK2 messenger RNA is not present in RIPK2sv1.

As used herein, “RIPK2sv1.1” refers to polynucleotides encoding RIPK2sv1.1 having an amino acid sequence set forth in SEQ ID NO 2. As used herein, “RIPK2sv1.2” refers to polynucleotides encoding RIPK2sv1.2 having an amino acid sequence set forth in SEQ ID NO 4. As used herein, “RIPK2sv2” refers to polynucleotides encoding RIPK2sv2 having an amino acid sequence set forth in SEQ ID NO 6.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human RIPK2sv1.1, RIPK2sv1.2, and RIPK2sv2 that are alternatively spliced isoforms of RIPK2, and to the amino acid sequences encoding these proteins. SEQ ID NO 1, SEQ ID NO 3, and SEQ ID NO 5 are polynucleotide sequences representing exemplary open reading frames that encode the RIPK2sv1.1, RIPK2sv1.2, and RIPK2sv2 proteins, respectively. SEQ ID NO 2 shows the polypeptide sequence of RIPK2sv1.1. SEQ ID NO 4 shows the polypeptide sequence of RIPK2sv1.2. SEQ ID NO 6 shows the polypeptide sequence of RIPK2sv2.

RIPK2sv1.1, RIPK2sv1.2, and RIPK2sv2 polynucleotide sequences encoding RIPK2sv1.1, RIPK2sv1.2, and RIPK2sv2 proteins, as exemplified and enabled herein include a number of specific, substantial and credible utilities. For example, RIPK2sv1.1, RIPK2sv1.2, and RIPK2sv2 encoding nucleic acids were identified in a mRNA sample obtained from a human source (see Example 1). Such nucleic acids can be used as hybridization probes to distinguish between cells that produce RIPK2sv1.1, RIPK2sv1.2, and RIPK2sv2 transcripts from human or non-human cells (including bacteria) that do not produce such transcripts. Similarly, antibodies specific for RIPK2sv1.1 or RIPK2sv2 can be used to distinguish between cells that express RIPK2sv1.1 or RIPK2sv2 from human or non-human cells (including bacteria) that do not express RIPK2sv1.1 or RIPK2sv2.

RIPK2 is an important drug target for the management of innate and adaptive immune function and inflammation responses (Kobayashi et. al., 2002; Chin et. al., 2002), the regulation of cell differentiation and apoptosis (Munz et. al., 2002; Inohara et. al., 1998; Thome et. al., 1998; McCarthy et. al, 1998), as well as diseases linked to the misregulation of NFkB such as rheumatoid arthritis, lupus, HIV-AIDS, influenza, and cancer (Baldwin, A. S., 1996, Annu. Rev. Immunol. 14, 649-681; May, et. al., 2000, Science 289, 1550-1554). Given the potential importance of RIPK2 activity to the therapeutic management of a wide array of diseases, it is of value to identify RIPK2 isoforms and identify RIPK2-ligand compounds that are isoform specific, as well as compounds that are effective ligands for two or more different RIPK2 isoforms. In particular, it may be important to identify compounds that are effective inhibitors of a specific RIPK2 isoform activity, yet do not bind to or interact with a plurality of different RIPK2 isoforms. Compounds that bind to or interact with multiple RIPK2 isoforms may require higher drug doses to saturate multiple RIPK2-isoform binding sites and thereby result in a greater likelihood of secondary non-therapeutic side effects. Furthermore, biological effects could also be caused by the interaction of a drug with the RIPK2sv1.1, RIPK2sv1.2, or RIPK2sv2 isoforms specifically. For the foregoing reasons, RIPK2sv1.1, RIPK2sv1.2, and RIPK2sv2 proteins represent useful compound binding targets and have utility in the identification of new RIPK2-ligands exhibiting a preferred specificity profile and having greater efficacy for their intended use.

In some embodiments, RIPK2sv1.1, RIPK2sv1.2, and RIPK2sv2 activity is modulated by a ligand compound to achieve one or more of the following: management of innate and adaptive immune function and inflammation responses, management of cell differentiation, regulation of cellular apoptosis, prevention or reduction of the risk of occurrence, or recurrence of disorders linked to NFkB misregulation including rheumatoid arthritis, septic shock, lupus, HIV-AIDS, viral infections, and cancer.

Compounds modulating RIPK2sv1.1, RIPK2sv1.2, or RIPK2sv2 include agonists, antagonists, and allosteric modulators. While not wishing to be limited to any particular theory of therapeutic efficacy, generally, but not always, RIPK2sv1.1, RIPK2sv1.2, or RIPK2sv2 compounds will be used to modulate the activity or expression of RIPK2. The expression level of RIPK2 has been shown to correlate with the level of cellular apoptosis. Increased expression of RIPK2 increases apoptosis in MCF7 cells (McCarthy et. al., 1998). In Schwann cells, increased expression of RIPK2 decreases apoptosis induced by nerve growth factor stimulation of the p75 receptor (Khursigara et. al., 2001). Thus, it is hypothesized that regulation of RIPK2 expression level or activity will modulate the level of cellular apoptosis in particular populations of cells, which is of therapeutic benefit in treating diseases in which regulation of apoptosis is critical such as AIDS, auto-immune diseases, neurogenerative diseases, cancer and viral infections. Increased expression of RIPK2 also results in increased activation of NFkB (Thome et. al., 1998; McCarthy et. al., 1998; Medzhitov et. al., 2000; Khursigara et. al., 2001; Inohara et. al., 2000), a transcription factor whose function has been implicated in diseases such as rheumatoid arthritis, lupus, HIV-AIDS, influenza, septic shock, atherosclerosis, and oncogenesis (reviewed in Baldwin, 1996). Therefore, agents that modulate RIPK2 activity may be used to achieve a therapeutic benefit for any disease or condition due to, or exacerbated by, abnormal levels of NFkB protein or its activity. RIPK2 deficient mice exhibit severe innate and adaptive immunity and inflammation response abnormalities (Kobayashi et. al., 2002; Chin et. al., 2002). Thus, compounds which affect the activity or regulation of RIPK2 may be used for immune intervention. Finally, RIPK2 expression level has been shown to be critical to the myogenic differentiation checkpoint (Munz et. al., 2002). It is hypothesized that compounds that modify the activity or expression of RIPK2 may regulate the proliferation and differentiation of cell populations.

RIPK2sv1.1, RIPK2sv1.2, or RIPK2sv2 activity can also be affected by modulating the cellular abundance of transcripts encoding RIPK2sv1.1, RIPK2sv1.2, or RIPK2sv2, respectively. Compounds modulating the abundance of transcripts encoding RIPK2sv1.1, RIPK2sv1.2, or RIPK2sv2 include a cloned polynucleotide encoding RIPK2sv1.1, RIPK2sv1.2, or RIPK2sv2, respectively, that can express RIPK2sv1.1, RIPK2sv1.2, or RIPK2sv2 in vivo, antisense nucleic acids targeted to RIPK2sv1.1, RIPK2sv1.2, or RIPK2sv2 transcripts, and enzymatic nucleic acids, such as ribozymes and siRNA, targeted to RIPK2sv1.1, RIPK2sv1.2, or RIPK2sv2 transcripts.

In some embodiments, RIPK2sv1.1, RIPK2sv1.2, or RIPK2sv2 activity is modulated to achieve a therapeutic effect upon diseases in which regulation of apoptosis, innate and adaptive immune responses and inflammation, cellular differentiation, and NFkB is desirable. For example, diseases which involve excessive or deficient apoptosis may be treated by modulating RIPK2sv1.1, RIPK2sv1.2, or RIPK2sv2 activities to inhibit or increase cellular apoptosis as required. In other embodiments, diseases which result from inadequate innate and adaptive immune or inflammation responses may be treated by increasing or otherwise modulating the activity of RIPK2. In other embodiments, diseases which result from irregular differentiation may be treated by decreasing or otherwise modulating the activity of RIPK2. In other embodiments, diseases which result from abnormal expression of NFkB may be treated by modulating RIPK2sv1.1, RIPK2sv1.2, or RIPK2sv2 to alter the activation of NFkB.

RIPK2sv1.1, RIPK2sv1.2, and RIPK2sv2 Nucleic Acids

RIPK2sv1.1 nucleic acids contain regions that encode for polypeptides comprising, consisting, or consisting essentially of SEQ ID NO 2. RIPK2sv1.2 nucleic acids contain regions that encode for polypeptides comprising, consisting, or consisting essentially of SEQ ID NO 4. RIPK2sv2 nucleic acids contain regions that encode for polypeptides comprising, consisting, or consisting essentially of SEQ ID NO 6. The RIPK2sv1.1, RIPK2sv1.2, and RIPK2sv2 nucleic acids have a variety of uses, such as use as a hybridization probe or PCR primer to identify the presence of RIPK2sv1.1, RIPK2sv1.2, or RIPK2sv2 nucleic acids, respectively; use as a hybridization probe or PCR primer to identify nucleic acids encoding for proteins related to RIPK2sv1.1, RIPK2sv1.2, or RIPK2sv2, respectively; and/or use for recombinant expression of RIPK2sv1.1, RIPK2sv1.2, or RIPK2sv2 polypeptides, respectively. In particular, RIPK2sv1.1 polynucleotides do not have the polynucleotide region that consists of exon 2 of the RIPK2 gene. RIPK2sv1.2 polynucleotides do not have the polynucleotide regions that consists of exon 1, exon 2, and the first 84 nucleotides of exon 3 of the RIPK2 gene. RIPK2sv2 polynucleotides do not have the polynucleotide region that consists of exon 8 of the RIPK2 gene.

Regions in RIPK2sv1.1, RIPK2sv1.2, or RIPK2sv2 nucleic acid that do not encode for RIPK2sv1.1, RIPK2sv1.2, or RIPK2sv2, or are not found in SEQ ID NO 1, SEQ ID NO 3, or SEQ ID NO 5, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding RIPK2sv1.1, RIPK2sv1.2, or RIPK2sv2 related proteins from different sources. Obtaining nucleic acids RIPK2sv1.1, RIPK2sv1.2, or RIPK2sv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

RIPK2sv1.1, RIPK2sv1.2, or RIPK2sv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

RIPK2sv1.1 and RIPK2sv2 Probes

Probes for RIPK2sv1.1 or RIPK2sv2 contain a region that can specifically hybridize to RIPK2sv1.1 or RIPK2sv2 target nucleic acids, respectively, under appropriate hybridization conditions and can distinguish RIPK2sv1.1 or RIPK2sv2 nucleic acids from each other and from non-target nucleic acids, in particular RIPK2 polynucleotides containing exons 2 and 8. Probes for RIPK2sv1.1 or RIPK2sv2 can also contain nucleic acid regions that are not complementary to RIPK2sv1.1 or RIPK2sv2 nucleic acids.

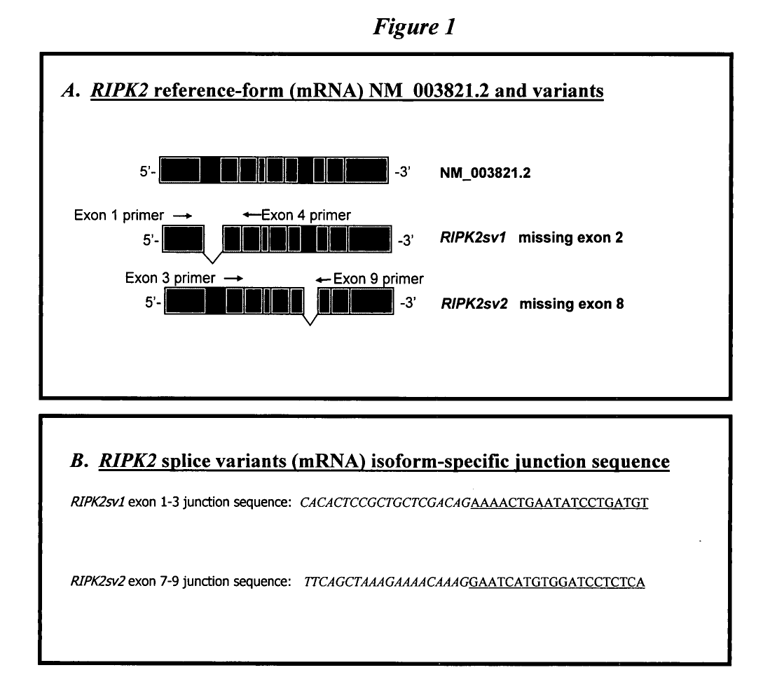

In embodiments where, for example, RIPK2sv1.1 or RIPK2sv2 polynucleotide probes are used in hybridization assays to specifically detect the presence of RIPK2sv1.1 or RIPK2sv2 polynucleotides in samples, the RIPK2sv1.1 or RIPK2sv2 polynucleotides comprise at least 20 nucleotides of the RIPK2sv1.1 or RIPK2sv2 sequence that correspond to the respective novel exon junction polynucleotide regions. In particular, for detection of RIPK2sv1.1, the probe comprises at least 20 nucleotides of the RIPK2sv1.1 sequence that corresponds to an exon junction polynucleotide created by the alternative splicing of exon 1 to exon 3 of the primary transcript of the RIPK2 gene (see FIGS. 1A and 1B). For example, the polynucleotide sequence: 5′ TGCTCGAC AGAAAACTGAAT 3′ [SEQ ID NO 7] represents one embodiment of such an inventive RIPK2sv1.1 polynucleotide wherein a first 10 nucleotide region is complementary and hybridizable to the 3′ end of exon 1 of the RIPK2 gene and a second 10 nucleotide region is complementary and hybridizable to the 5′ end of exon 3 of the RIPK2 gene (see FIG. 1B).

In another embodiment, for detection of RIPK2sv2, the probe comprises at least 20 nucleotides of the RIPK2sv2 sequence that corresponds to an exon junction polynucleotide created by the alternative splicing of exon 7 to exon 9 of the primary transcript of the RIPK2 gene (see FIGS. 1A and 1B). For example, the polynucleotide sequence: 5′ GAAAACAAAGGAA TCATGTG 3′ [SEQ ID NO 8] represents one embodiment of such an inventive RIPK2sv2 polynucleotide wherein a first 10 nucleotide region is complementary and hybridizable to the 3′ end of exon 7 of the RIPK2 gene and a second 10 nucleotide region is complementary and hybridizable to the 5′ end of exon 9 of the RIPK2 gene (see FIG. 1B).

In some embodiments, the first 20 nucleotides of a RIPK2sv1.1 probe comprise a first continuous region of 5 to 15 nucleotides that is complementary and hybridizable to the 3′ end of exon 1 and a second continuous region of 5 to 15 nucleotides that is complementary and hybridizable to the 5′ end of exon 3. In some embodiments, the first 20 nucleotides of a RIPK2sv2 probe comprise a first continuous region of 5 to 15 nucleotides that is complementary and hybridizable to the 3′ end of exon 7 and a second continuous region of 5 to 15 nucleotides that is complementary and hybridizable to the 5′ end of exon 9.

In other embodiments, the RIPK2sv1.1 or RIPK2sv2 polynucleotide comprises at least 40, 60, 80 or 100 nucleotides of the RIPK2sv1.1 or RIPK2sv2 sequence, respectively, that correspond to a junction polynucleotide region created by the alternative splicing of exon 1 to exon 3 in the case of RIPK2sv1.1, or in the case of RIPK2sv2, by the alternative splicing of exon 7 to exon 9 of the primary transcript of the RIPK2 gene. In embodiments involving RIPK2sv1.1, the RIPK2sv1.1 polynucleotide is selected to comprise a first continuous region of at least 5 to 15 nucleotides that is complementary and hybridizable to the 3′ end of exon 1 and a second continuous region of at least 5 to 15 nucleotides that is complementary and hybridizable to the 5′ end of exon 3. Similarly, in embodiments involving RIPK2sv2, the RIPK2sv2 polynucleotide is selected to comprise a first continuous region of at least 5 to 15 nucleotides that is complementary and hybridizable to the 3′ end of exon 7 and a second continuous region of at least 5 to 15 nucleotides that is complementary and hybridizable to the 5′ end of exon 9. As will be apparent to a person of skill in the art, a large number of different polynucleotide sequences from the region of the exon 1 to exon 3 splice junction and the exon 7 to exon 9 splice junction may be selected which will, under appropriate hybridization conditions, have the capacity to detectably hybridize to RIPK2sv1.1 or RIPK2sv2 polynucleotides, respectively, and yet will hybridize to a much less extent or not at all to RIPK2 isoform polynucleotides wherein exon 1 is not spliced to exon 3 or wherein exon 7 is not spliced to exon 9, respectively.

Preferably, non-complementary nucleic acid that is present has a particular purpose such as being a reporter sequence or being a capture sequence. However, additional nucleic acid need not have a particular purpose as long as the additional nucleic acid does not prevent the RIPK2sv1.1 or RIPK2sv2 nucleic acid from distinguishing between target polynucleotides, e.g., RIPK2sv1.1 or RIPK2sv2 polynucleotides, and non-target polynucleotides, including, but not limited to RIPK2 polynucleotides not comprising the exon 1 to exon 3 splice junction or the exon 7 to exon 9 splice junctions found in RIPK2sv1.1 or RIPK2sv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5 to 20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Recombinant Expression

RIPK2sv1.1, RIPK2sv1.2, or RIPK2sv2 polynucleotides, such as those comprising SEQ ID NO 1, SEQ ID NO 3, or SEQ ID NO 5, respectively, can be used to make RIPK2sv1.1, RIPK2sv1.2, or RIPK2sv2 polypeptides, respectively. In particular, RIPK2sv1.1, RIPK2sv1.2, or RIPK2sv2 polypeptides can be expressed from recombinant nucleic acids in a suitable host or in vitro using a translation system. Recombinantly expressed RIPK2sv1.1, RIPK2sv1.2, or RIPK2sv2 polypeptides can be used, for example, in assays to screen for compounds that bind RIPK2sv1.1, RIPK2sv1.2, or RIPK2sv2, respectively. Alternatively, RIPK2sv1.1, RIPK2sv1.2, or RIPK2sv2 polypeptides can also be used to screen for compounds that bind to one or more RIPK2 isoforms, but do not bind to RIPK2sv1.1, RIPK2sv1.2, or RIPK2sv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M (TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 1, SEQ ID NO 3, or SEQ ID NO 5 to take into account codon usage of the host. Codon usage of different organisms is well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

RIPK2sv1.1, RIPK2sv1.2, and RIPK2sv2 POLYPEPTIDES

RIPK2sv1.1 polypeptides contain an amino acid sequence comprising, consisting or consisting essentially of SEQ ID NO 2. RIPK2sv1.2 polypeptides contain an amino acid sequence comprising, consisting or consisting essentially of SEQ ID NO 4. RIPK2sv2 polypeptides contain an amino acid sequence comprising, consisting or consisting essentially of SEQ ID NO 6. RIPK2sv1.1, RIPK2sv1.2, or RIPK2sv2 polypeptides have a variety of uses, such as providing a marker for the presence of RIPK2sv1.1, RIPK2sv1.2, or RIPK2sv2, respectively; use as an immunogen to produce antibodies binding to RIPK2sv1.1, RIPK2sv1.2, or RIPK2sv2, respectively; use as a target to identify compounds binding selectively to RIPK2sv1.1, RIPK2sv1.2, or RIPK2sv2, respectively; or use in an assay to identify compounds that bind to one or more isoforms of RIPK2 but do not bind to or interact with RIPK2sv1. 1, RIPK2sv1.2, or RIPK2sv2, respectively.

In chimeric polypeptides containing one or more regions from RIPK2sv1.1, RIPK2sv1.2, or RIPK2sv2 and one or more regions not from RIPK2sv1.1, RIPK2sv1.2, or RIPK2sv2, respectively, the region(s) not from RIPK2sv1.1, RIPK2sv1.2, or RIPK2sv2, respectively, can be used, for example, to achieve a particular purpose or to produce a polypeptide that can substitute for RIPK2sv1.1, RIPK2sv1.2, or RIPK2sv2, or fragments thereof. Particular purposes that can be achieved using chimeric RIPK2sv1.1, RIPK2sv1.2, or RIPK2sv2 polypeptides include providing a marker for RIPK2sv1.1, RIPK2sv1.2, or RIPK2sv2 activity, respectively, altering cellular differentiation, modulating the innate and adaptive immune and inflammatory responses, modulating the activation of NFkB, and regulating cellular apoptosis.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional RIPK2sv1.1, RIPK2sv1.2, and RIPK2sv2

Functional RIPK2sv1.1, RIPK2sv1.2, and RIPK2sv2 are different protein isoforms of RIPK2. The identification of the amino acid and nucleic acid sequences of RIPK2sv1.1, RIPK2sv1.2, or RIPK2sv2 provide tools for obtaining functional proteins related to RIPK2sv1.1, RIPK2sv1.2, or RIPK2sv2, respectively, from other sources, for producing RIPK2sv1.1, RIPK2sv1.2, or RIPK2sv2 chimeric proteins, and for producing functional derivatives of SEQ ID NO 2, SEQ ID NO 4, or SEQ ID NO 6.

RIPK2sv1.1, RIPK2sv1.2, or RIPK2sv2 polypeptides can be readily identified and obtained based on their sequence similarity to RIPK2sv1.1 (SEQ ID NO 2), RIPK2sv1.2 (SEQ ID NO 4), or RIPK2sv2 (SEQ ID NO 6), respectively. In particular, RIPK2sv1.1 lacks the amino acids encoded by exon 2 of the RIPK2 gene. The deletion of exon 2 and the splicing of exon 1 to exon 3 of the RIPK2 hnRNA transcript results in a shift of the protein reading frame at the exon 1 to exon 3 splice junction, thereby creating an amino terminal peptide region that is unique to the RIPK2sv1.1 polypeptide as compared to other known RIPK2 isoforms. The frame shift creates a premature termination codon thirty-two nucleotides downstream of the exon 1/exon 3 splice junction. Thus, the RIPK2sv1.1 polypeptide is also lacking the amino acids encoded by the nucleotides downstream of the premature stop codon, including the C-terminal CARD domain and most of the N-terminal kinase domain. The RIPK2sv1.2 polypeptide initiates at an AUG located 257 nucleotides downstream of the initiation AUG of the RIPK2 reference sequence NM_{—}003821.2. Initiation at a downstream AUG of a bicistronic RNA is a fairly common event and can be associated with disease (Meijer and Thomas, 2002 Biochem. J., 367:1-11; Kozak, 2002, Mammalian Genome 13:401-410). The RIPK2sv1.2 polypeptide is translated in a different reading frame compared to other RIPK2 isoforms and therefore has a completely unique amino acid sequence. The RIPK2sv2 polypeptide lacks the amino acids encoded by exon 8 of the RIPK2 gene. The deletion of exon 8 does not result in a reading frame shift. Thus, the RIPK2sv2 polypeptide lacks only the amino acid region encoded by exon 8 and has a unique amino acid sequence at the new exon 7/exon 9 splice junction. The RIPK2sv2 polypeptide includes the N-terminal kinase domain and the C-terminal CARD domain but has a shortened interdomain region.

Both the amino acid and nucleic acid sequences of RIPK2sv1.1, RIPK2sv1.2, or RIPK2sv2 can be used to help identify and obtain RIPK2sv1.1, RIPK2sv1.2, or RIPK2sv2 polypeptides, respectively. For example, SEQ ID NO 1 can be used to produce degenerative nucleic acid probes or primers for identifying and cloning nucleic acid polynucleotides encoding for a RIPK2sv1.1 polypeptide. In addition, polynucleotides comprising, consisting, or consisting essentially of SEQ ID NO 1 or fragments thereof, can be used under conditions of moderate stringency to identify and clone nucleic acids encoding RIPK2sv1.1 polypeptides from a variety of different organisms. The same methods can also be performed with polynucleotides comprising, consisting, or consisting essentially of SEQ ID NO 3, or SEQ ID NO 5, or fragments thereof, to identify and clone nucleic acids encoding RIPK2sv1.2 and RIPK2sv2, respectively.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with RIPK2sv1.1, RIPK2sv1.2, or RIPK2sv2 obtained from a particular source, derivatives can be produced. Such derivatives include polypeptides with amino acid substitutions, additions and deletions. Changes to RIPK2sv1.1, RIPK2sv1.2, or RIPK2sv2 to produce a derivative having essentially the same properties should be made in a manner not altering the tertiary structure of RIPK2sv1.1, RIPK2sv1.2, or RIPK2sv2, respectively.

Differences in naturally occurring amino acids are due to different R groups. An R group effects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

RIPK2sv1.1 and RIPK2sv2 Antibodies

Antibodies recognizing RIPK2sv1.1 or RIPK2sv2 can be produced using a polypeptide containing SEQ ID NO 2 in the case of RIPK2sv1.1, or SEQ ID NO 6 in the case of RIPK2sv2, respectively, or a fragment thereof, as an immunogen. Preferably, a RIPK2sv1.1 polypeptide used as an immunogen consists of a polypeptide of SEQ ID NO 2 or a SEQ ID NO 2 fragment having at least 10 contiguous amino acids in length corresponding to the polynucleotide region representing the junction resulting from the splicing of exon 1 to exon 3 of the RIPK2 gene. Preferably, a RIPK2sv2 polypeptide used as an immunogen consists of a polypeptide derived from SEQ ID NO 6 or a SEQ ID NO 6 fragment, having at least 10 contiguous amino acids in length corresponding to a polynucleotide region representing the junction resulting from the splicing of exon 7 to exon 9 of the RIPK2 gene.

In some embodiments where, for example, RIPK2sv1.1 polypeptides are used to develop antibodies that bind specifically to RIPK2sv1.1 and not to other isoforms of RIPK2, the RIPK2sv1.1 polypeptides comprise at least 10 amino acids of the RIPK2sv1.1 polypeptide sequence corresponding to a junction polynucleotide region created by the alternative splicing of exon 1 to exon 3 of the primary transcript of the RIPK2 gene (see FIG. 1). For example, the amino acid sequence: amino terminus-TPLLDRKLNI-carboxy terminus [SEQ ID NO 9] represents one embodiment of such an inventive RIPK2sv1.1 polypeptide wherein a first 5 amino acid region is encoded by nucleotide sequence at the 3′ end of exon 1 of the RIPK2 gene and a second 5 amino acid region is encoded by the nucleotide sequence directly after the novel splice junction. Preferably, at least 10 amino acids of the RIPK2sv1.1 polypeptide comprises a first continuous region of 2 to 8 amino acids that is encoded by nucleotides at the 3′ end of exon 1 and a second continuous region of 2 to 8 amino acids that is encoded by nucleotides at the 5′ end of exon 3.

In other embodiments where, for example, RIPK2sv2 polypeptides are used to develop antibodies that bind specifically to RIPK2sv2 and not to other RIPK2 isoforms, the RIPK2sv2 polypeptides comprise at least 10 amino acids of the RIPK2sv2 polypeptide sequence corresponding to a junction polynucleotide region created by the alternative splicing of exon 7 to exon 9 of the primary transcript of the RIPK2 gene (see FIG. 1). For example, the amino acid sequence: amino terminus-LKKTKESCGS-carboxy terminus [SEQ ID NO 10], represents one embodiment of such an inventive RIPK2sv2 polypeptide wherein a first 5 amino acid region is encoded by a nucleotide sequence at the 3′ end of exon 7 of the RIPK2 gene and a second 5 amino acid region is encoded by a nucleotide sequence directly after the novel splice junction. Preferably, at least 10 amino acids of the RIPK2sv2 polypeptide comprises a first continuous region of 2 to 8 amino acids that is encoded by nucleotides at the 3′ end of exon 7 and a second continuous region of 2 to 8 amino acids that is encoded by nucleotides at the 5′ end of exon 9.

In other embodiments, RIPK2sv1.1-specific antibodies are made using a RIPK2sv1.1 polypeptide that comprises at least 20, 30, 40 or 50 amino acids of the RIPK2sv1.1 sequence that corresponds to a junction polynucleotide region created by the alternative splicing of exon 1 to exon 3 of the primary transcript of the RIPK2 gene. In each case the RIPK2sv1.1 polypeptides are selected to comprise a first continuous region of at least 5 to 15 amino acids that is encoded by nucleotides at the 3′ end of exon 1 and a second continuous region of 5 to 15 amino acids that is encoded by nucleotides directly after the novel splice junction.

In other embodiments, RIPK2sv2-specific antibodies are made using a RIPK2sv2 polypeptide that comprises at least 20, 30, 40 or 50 amino acids of the RIPK2sv2 sequence that corresponds to a junction polynucleotide region created by the alternative splicing of exon 7 to exon 9 of the primary transcript of the RIPK2 gene. In each case the RIPK2sv2 polypeptides are selected to comprise a first continuous region of at least 5 to 15 amino acids that is encoded by nucleotides at the 3′ end of exon 7 and a second continuous region of 5 to 15 amino acids that is encoded by nucleotides directly after the novel splice junction.

Antibodies to RIPK2sv1.1 or RIPK2sv2 have different uses, such as to identify the presence of RIPK2sv1.1 or RIPK2sv2, respectively, and to isolate RIPK2sv1.1 or RIPK2sv2 polypeptides, respectively. Identifying the presence of RIPK2sv1.1 can be used, for example, to identify cells producing RIPK2sv1.1. Such identification provides an additional source of RIPK2sv1.1 and can be used to distinguish cells known to produce RIPK2sv1.1 from cells that do not produce RIPK2sv1.1. For example, antibodies to RIPK2sv1.1 can distinguish human cells expressing RIPK2sv1.1 from human cells not expressing RIPK2sv1.1 or non-human cells (including bacteria) that do not express RIPK2sv1.1. Such RIPK2sv1.1 antibodies can also be used to determine the effectiveness of RIPK2sv1.1 ligands, using techniques well known in the art, to detect and quantify changes in the protein levels of RIPK2sv1.1 in cellular extracts, and in situ immunostaining of cells and tissues. In addition, the same above-described utilities also exist for RIPK2sv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

RIPK2sv1.1 and RIPK2sv2 Binding Assay

Compounds which bind to RIPK2 isoforms may modulate RIPK2 function. These compounds may, for example, affect the ability of RIPK2 to interact with protein binding partners or may alter the serine-threonine kinase activity of RIPK2. Polypeptides comprising a CARD domain may also alter the function of RIPK2. For example, the CARD domain of Caspase-1 has a greater affinity for the CARD domain of ICEBERG than for the CARD domain of RIPK2 (Humke et. al., 2000). ICEBERG is therefore able to displace RIPK2 bound to Caspase-1 through its CARD domain. Protein-protein interactions mediated by CARD domains have also been reported to be disrupted by nitric oxide (NO) (Zech et. al., 2003, Biochem J. 371 (Part 3): 1055-64). Compounds may also affect the function of RIPK2 by altering its serine-theonine kinase activity. Methods for measuring the kinase activity of RIPK2 isoforms have been described previously (Inohara et. al., 1998; Thome et. al., 1998; McCarthy et. al., 1998; Navas et. al., 1999). Methods for screening for compounds that modulate serine-threonine kinase activity have been disclosed (US2003/0134310A1; WO 02/14542). A person skilled in the art should be able to use these methods to screen RIPK2sv1.1 or RIPK2sv2 polypeptides for compounds that bind to, and in some cases functionally alter, each respective RIPK2 isoform protein.

RIPK2sv1.1, RIPK2sv2, or fragments thereof, can be used in binding studies to identify compounds binding to or interacting with RIPK2sv1.1, RIPK2sv2, or fragments thereof, respectively. In one embodiment, the RIPK2sv1.1, or a fragment thereof, can be used in binding studies with RIPK2 isoform protein, or a fragment thereof, to identify compounds that: bind to or interact with RIPK2sv1.1 and other RIPK2 isoforms; bind to or interact with one or more other RIPK2 isoforms and not with RIPK2sv1.1. A similar series of compound screens can, of course, also be performed using RIPK2sv2 rather than, or in addition to, RIPK2sv1.1. Such binding studies can be performed using different formats including competitive and non-competitive formats. Further competition studies can be carried out using additional compounds determined to bind to RIPK2sv1.1, RIPK2sv2 or other RIPK2 isoforms.

The particular RIPK2sv1.1 or RIPK2sv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using RIPK2sv1.1 expressed from a recombinant nucleic acid. Alternatively, recombinantly expressed RIPK2sv1.1 consists of the SEQ ID NO 2 amino acid sequence. In addition, binding studies are performed using RIPK2sv2 expressed from a recombinant nucleic acid. Alternatively, recombinantly expressed RIPK2sv2 consists of the SEQ ID NO 6 amino acid sequence.

Binding assays can be performed using individual compounds or preparations containing different numbers of compounds. A preparation containing different numbers of compounds having the ability to bind to RIPK2sv1.1 or RIPK2sv2 can be divided into smaller groups of compounds that can be tested to identify the compound(s) binding to RIPK2sv1.1 or RIPK2sv2, respectively.

Binding assays can be performed using recombinantly produced RIPK2sv1.1 or RIPK2sv2 present in different environments. Such environments include, for example, cell extracts and purified cell extracts containing a RIPK2sv1.1 or RIPK2sv2 recombinant nucleic acid; and also include, for example, the use of a purified RIPK2sv1.1 or RIPK2sv2 polypeptide produced by recombinant means which is introduced into different environments.

In one embodiment of the invention, a binding method is provided for screening for a compound able to bind selectively to RIPK2sv1.1. The method comprises the steps: providing a RIPK2sv1.1 polypeptide comprising SEQ ID NO 2; providing a RIPK2 isoform polypeptide that is not RIPK2sv1.1; contacting the RIPK2sv1.1 polypeptide and the RIPK2 isoform polypeptide that is not RIPK2sv1.1 with a test preparation comprising one or more test compounds; and then determining the binding of the test preparation to the RIPK2sv1.1 polypeptide and to the RIPK2 isoform polypeptide that is not RIPK2sv1.1, wherein a test preparation that binds to the RIPK2sv1.1 polypeptide, but does not bind to RIPK2 isoform polypeptide that is not RIPK2sv1.1, contains one or more compounds that selectively binds to RIPK2sv1.1.

In another embodiment of the invention, a binding method is provided for screening for a compound able to bind selectively to RIPK2sv2. The method comprises the steps: providing a RIPK2sv2 polypeptide comprising SEQ ID NO 6; providing a RIPK2 isoform polypeptide that is not RIPK2sv2; contacting the RIPK2sv2 polypeptide and the RIPK2 isoform polypeptide that is not RIPK2sv2 with a test preparation comprising one or more test compounds; and then determining the binding of the test preparation to the RIPK2sv2 polypeptide and to the RIPK2 isoform polypeptide that is not RIPK2sv2, wherein a test preparation that binds to the RIPK2sv2 polypeptide, but does not bind to RIPK2 isoform polypeptide that is not RIPK2sv2, contains one or more compounds that selectively binds to RIPK2sv2.

In another embodiment of the invention, a binding method is provided to screen for a compound able to bind selectively to a RIPK2 isoform polypeptide that is not RIPK2sv1.1. The method comprises the steps: providing a RIPK2sv1.1 polypeptide comprising SEQ ID NO 2; providing a RIPK2 isoform polypeptide that is not RIPK2sv1.1; contacting the RIPK2sv1.1 polypeptide and the RIPK2 isoform polypeptide that is not RIPK2sv1.1 with a test preparation comprising one or more test compounds; and then determining the binding of the test preparation to the RIPK2sv1.1 polypeptide and the RIPK2 isoform polypeptide that is not RIPK2sv1.1, wherein a test preparation that binds the RIPK2 isoform polypeptide that is not RIPK2sv1.1, but does not bind the RIPK2sv1.1, contains a compound that selectively binds the RIPK2 isoform polypeptide that is not RIPK2sv1.1. Alternatively, the above method can be used to identify compounds that bind selectively to a RIPK2 isoform polypeptide that is not RIPK2sv2 by performing the method with RIPK2sv2 protein comprising SEQ ID NO 6.

The above-described selective binding assays can also be performed with a polypeptide fragment of RIPK2sv1.1 or RIPK2sv2, wherein the polypeptide fragment comprises at least 10 consecutive amino acids that are coded by a nucleotide sequence that bridges the junction created by the splicing of the 3′ end of exon 1 to the 5′ end of exon 3 in the case of RIPK2sv1.1 or by the splicing of the 3′ end of exon 7 to the 5′ end of exon 9, in the case of RIPK2sv2. Similarly, the selective binding assays may also be performed using a polypeptide fragment of an RIPK2 isoform polypeptide that is not RIPK2sv1.1 or RIPK2sv2, wherein the polypeptide fragment comprises at least 10 consecutive amino acids that are coded by: a) a nucleotide sequence that is contained within exon 2 or 8 of the RIPK2 gene; or b) a nucleotide sequence that bridges the junction created by the splicing of the 3′ end of exon 1 to the 5′ end of exon 3 or the splicing of the 3′ end of exon 7 to the 5′ end of exon 9 of the RIPK2 gene.

RIPK2 Functional Assays

RIPK2 functions in receptor signaling pathways initiated upon activation of several tumor necrosis factor family receptors including TNFR-1, CD40 and Fas (CD-95/APO-1) as well as the p75 receptor (Inohara et. al., 1998; McCarthy et. al., 1998; Khursigara et. al., 2001). RIPK2 expression level influences NFkB and Jun N-terminal kinase activation, apoptosis, adaptive and innate immunity and inflammation, and cellular differentiation (Thome et. al., 1998; McCarthy et. al., 1998; Medzhitov et. al., 2000; Inohara et. al., 2000; Khursigara et. al., 2001; Chin et. al., 2002; Kobayashi et. al., 2002; Munz et. al., 2002). RIPK2 has a C-terminal CARD domain that mediates protein-protein interactions as well as an N-terminal kinase domain that autophosphorylates RIPK2 and phosphorylates other proteins such as ERK1 and ERK2 (Thome et. al., 1998; Inohara et. al., 1998; McCarthy et. al., 1998; Navas et. al., 1999). The CARD domain mediates RIPK2 association with pro-Caspase-1 and the p75 receptor (Thome et. al., 1998; Humke et. al., 2000; Khursigara et. al., 2001). RIPK2 also physically associates with CLARP (a caspase related protein that interacts with FADD and Caspase-8), TRAF1, TRAF2, TRAF5, TRAF6, Raf1, and IKK-γ (a regulatory subunit of the IKK complex that is essential for induction of NFkB activation) (Inohara et. al., 1998; Thome et. al., 1998; McCarthy et. al., 1998; Navas et. al., 1999; Inohara et. al., 2000). The identification of RIPK2sv1.1 and RIPK2sv2 as splice variants of RIPK2 provides a means for screening for compounds that bind to RIPK2sv1.1 and/or RIPK2sv2 protein thereby altering the ability of the RIPK2sv1.1 and/or RIPK2sv2 polypeptide to bind to its protein binding partners or to phosphorylate itself or other proteins. Assays involving a functional RIPK2sv1.1 or RIPK2sv2 polypeptide can be employed for different purposes, such as selecting for compounds active at RIPK2sv1.1 or RIPK2sv2; evaluating the ability of a compound to effect the phosphorylation of, kinase activity of, or binding affinity for RIPK2 protein binding partners of each respective splice variant polypeptide; and mapping the activity of different RIPK2sv1.1 and RIPK2sv2 regions. RIPK2sv1.1 and RIPK2sv2 activity can be measured using different techniques such as: detecting a change in the intracellular conformation of RIPK2sv1.1 or RIPK2sv2; detecting a change in the intracellular location of RIPK2sv1.1 or RIPK2sv2; detecting the amount of binding of RIPK2sv1.1 or RIPK2sv2 to RIPK2 protein binding partners; or by measuring the level of protein kinase activity of the RIPK2 isoform.

Recombinantly expressed RIPK2sv1.1 and RIPK2sv2 can be used to facilitate the determination of whether a compound is active at RIPK2sv1.1 and RIPK2sv2. For example, RIPK2sv1.1 and RIPK2sv2 can be expressed by an expression vector in a cell line and used in a co-culture growth assay, such as described in U.S. Pat. No. 6,518,035, to identify compounds that alter the growth of the cell expressing RIPK2sv1.1 and RIPK2sv2 from the expression vector as compared to the same cell line but lacking the RIPK2sv1.1 and RIPK2sv2 expression vector. Alternatively, determination of whether a compound's activity on a cell is dependent upon the presence of RIPK2sv1.1 and RIPK2sv2 can also be done using gene expression profile analysis methods as described, for example, in U.S. Pat. No. 6,324,479.

Techniques for measuring protein kinase activity (Navas et. al., 1999) as well as techniques for measuring cell death resulting from apoptosis (McCarthy et. al., 1998; Inohara et. al., 1998; Khursigara et. al., 2001) are well known in the art. Methods for measuring NFkB activation based on a NFkB luciferase construct have also been described (McCarthy et. al., 1998; Inohara et. al., 1998; Khursigara et. al., 2001; Thome et. al., 1998). Munz et. al. (2002) report methods for measuring cell proliferation and differentiation in response to RIPK2 expression. The method involves pulse labeling cells with BrdU and assessing the percentage of proliferating cells by staining cells with an anti-BrdU antibody. Competitive protein binding assays using RIPK2 have also been described which assess the relative affinity of RIPK2 for its protein binding partners (Humke et. al., 2000). A variety of other assays have been used to investigate the properties of RIPK2 and therefore would also be applicable to the measurement of RIPK2sv1.1 or RIPK2sv2 functions.

RIPK2sv1.1 or RIPK2sv2 functional assays can be performed using cells expressing RIPK2sv1.1 or RIPK2sv2 at a high level. These proteins will be contacted with individual compounds or preparations containing different compounds. A preparation containing different compounds where one or more compounds affect RIPK2sv1.1 or RIPK2sv2 in cells over-producing RIPK2sv1.1 or RIPK2sv2 as compared to control cells containing expression vector lacking RIPK2sv1.1 or RIPK2sv2 coding sequences, can be divided into smaller groups of compounds to identify the compound(s) affecting RIPK2sv1.1 or RIPK2sv2 activity, respectively.

RIPK2sv1.1 or RIPK2sv2 functional assays can be performed using recombinantly produced RIPK2sv1.1 or RIPK2sv2 present in different environments. Such environments include, for example, cell extracts and purified cell extracts containing RIPK2sv1.1 or RIPK2sv2 expressed from recombinant nucleic acid; and the use of a purified RIPK2sv1.1 or RIPK2sv2 produced by recombinant means that is introduced into a different environment suitable for measuring binding or kinase activity.

MODULATING RIPK2sv1.1, RIPK2sv1.2, and RIPK2sv2 EXPRESSION

RIPK2sv1.1, RIPK2sv1.2, or RIPK2sv2 expression can be modulated as a means for increasing or decreasing RIPK2sv1.1, RIPK2sv1.2, or RIPK2sv2 activity, respectively. Such modulation includes inhibiting the activity of nucleic acids encoding the RIPK2 isoform target to reduce RIPK2 isoform protein or polypeptide expression, or supplying RIPK2 nucleic acids to increase the level of expression of the RIPK2 target polypeptide thereby increasing RIPK2 activity.

Inhibition of RIPK2sv1.1, RIPK2sv1.2, and RIPK2sv2 Activity

RIPK2sv1.1, RIPK2sv1.2, or RIPK2sv2 nucleic acid activity can be inhibited using nucleic acids recognizing RIPK2sv1.1, RIPK2sv1.2, or RIPK2sv2 nucleic acid and affecting the ability of such nucleic acid to be transcribed or translated. Inhibition of RIPK2sv1.1, RIPK2sv1.2, or RIPK2sv2 nucleic acid activity can be used, for example, in target validation studies.

A preferred target for inhibiting RIPK2sv1.1, RIPK2sv1.2, or RIPK2sv2 is mRNA stability and translation. The ability of RIPK2sv1, RIPK2sv1.2, or RIPK2sv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Anti-sense oligonucleotides designed to inhibit RIPK2 have been described in U.S. Pat. No. 6,426,221 B1. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing RIPK2sv1.1, RIPK2sv1.2, and RIPK2sv2 Expression

Nucleic acids encoding for RIPK2sv1.1, RIPK2sv1.2, or RIPK2sv2 can be used, for example, to cause an increase in RIPK2 activity or to create a test system (e.g., a transgenic animal) for screening for compounds affecting RIPK2sv1.1, RIPK2sv1.2, or RIPK2sv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

Identification of RIPK2sv1 and RIPK2sv2 Using Microarrays

To identify variants of the “normal” splicing of the exon regions encoding RIPK2, an exon junction microarray, comprising probes complementary to each splice junction resulting from splicing of the 11 exon coding sequences in RIPK2 heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least two of the exon junctions of RIPK2 mRNA were altered in some of the tissues examined, suggesting the presence of RIPK2 splice variant mRNA populations. Reverse transcription and polymerase chain reaction (RT-PCR) were then performed using oligonucleotide primers complementary to either exons 1 and 4 or to exons 3 and 9 to confirm the exon junction array results and to allow the sequence structure of the splice variants to be determined.

### Example 2

Confirmation of RIPK2sv1 and RIPK2sv2 Using RT-PCR

The structure of RIPK2 mRNA in the region corresponding to exons 1 to 9 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 1, exon 4, exon 3, and exon 9 of the reference exon coding sequences in RIPK2 (NM_{—}003821.2). Based upon the nucleotide sequence of RIPK2 mRNA, the RIPK2 exon 1 and exon 4 primer set (hereafter RIPK2,₁₋₄ primer set) was expected to amplify a 485 base pair amplicon representing the “reference” RIPK2 mRNA region; the RIPK2 exon 3 and exon 9 primer set (hereafter RIPK2₃₋₉ primer set) was expected to amplify a 723 base pair amplicon representing the “reference” RIPK2 mRNA region. The RIPK2 exon 1 forward primer has the sequence: 5′ CTGCCCACCATTCCC TACCACAAACT 3′ [SEQ ID NO 11]; the RIPK2 exon 4 reverse primer has the sequence: 5′ CGC CACTTTGATAAACCAAAATCTGCAA 3′ [SEQ ID NO 12]; the RIPK2 exon 3 forward primer has the sequence: 5′ ATATCCTGATGTTGCTTGGCCATTGAGA 3′ [SEQ ID NO 13]; and the RIPK2 exon 9 reverse primer has the sequence: 5′ TCATGGAGCTGAGAGGATCCACATGA TT 3′ [SEQ ID NO 14].

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

One different RT-PCR amplicon was obtained from human mRNA samples using the RIPK2₁₋₄ primer set (data not shown). Every human tissue and cell line assayed exhibited the expected amplicon size of 485 base pairs for normally spliced RIPK2 mRNA. However, in addition to the expected RIPK2 amplicon of 485 base pairs, all cell lines assayed also exhibited an amplicon of 331 base pairs. One different amplicon was also obtained from human mRNA samples using the RIPK2₃₋₉ primer set (data not shown). Every human tissue and cell line assayed, except heart, kidney, adrenal gland, ileocecum, and lung carcinoma (A549), exhibited the expected amplicon size of 723 base pairs for normally spliced RIPK2 mRNA. In addition to the expected RIPK2 amplicon of 723 base pairs, brain and fetal brain samples showed an additional amplicon of 633 base pairs. The tissues in which RIPK2sv1 and RIPK2sv2 mRNAs were detected are listed in Table 1:

TABLE 1

| Sample | RIPK2sv1 | RIPK2sv2 |
|---|---|---|
| Heart | x | |
| Kidney | x | |
| Liver | x | |
| Brain | x | x |
| Placenta | x | |
| Lung | x | |
| Fetal Brian | x | x |
| Leukemia Promyelocytic (HL-60) | x | |
| Adrenal Gland | x | |
| Fetal Liver | x | |
| Salivary Gland | x | |
| Pancreas | x | |
| Skeletal Muscle | x | |
| Brain Cerebellum | x | |
| Stomach | x | |
| Trachea | x | |
| Thyroid | x | |
| Bone Marrow | x | |
| Brain Amygdala | x | |
| Brain Caudate Nucleus | x | |
| Brain Corpus Callosum | x | |
| Ileocecum | x | |
| Lymphoma Burkitt's (Raji) | x | |
| Spinal Cord | x | |
| Lymph Node | X | |
| Fetal Kidney | x | |
| Uterus | x | |
| Spleen | x | |
| Brain Thalamus | x | |
| Fetal Lung | x | |
| Testis | x | |
| Melanoma (G361) | x | |
| Lung Carcinoma (A549) | x | |
| Adrenal Medula, normal | x | |
| Brain, Cerebral Cortex, normal; | x | |
| Descending Colon, normal | x | |
| Prostate | x | |
| Duodenum, normal | x | |
| Epididymus, normal | x | |
| Brain, Hippocamus, normal | x | |
| Ileum, normal | x | |
| Interventricular Septum, normal | x | |
| Jejunum, normal | x | |
| Rectum, normal | x | |

Sequence analysis of the about 331 base pair amplicon revealed that this amplicon form results from the splicing of exon 1 of the RIPK2 hnRNA to exon 3; that is, exon 2 coding sequence is completely absent. Sequence analysis of the about 633 base pair amplicon revealed that this amplicon form results from the splicing of exon 7 of the RIPK2 hnRNA to exon 9; that is, exon 8 coding sequence is completely absent. Thus, the RT-PCR results confirmed the junction probe microarray data reported in Example 1, which suggested that RIPK2 MRNA is composed of a mixed population of molecules wherein in at least two of the RIPK2 mRNA splice junctions are altered.

### Example 3

Cloning of RIPK2sv1.1, RIPK2sv1.2, and RIPK2sv2

Microarray and RT-PCR data indicate that in addition to the normal RIPK2 reference mRNA sequence, NM_{—}003821.2, encoding RIPK2 protein, NP_{—}003812, two novel splice variant forms of RIPK2 mRNA also exist in many tissues.

Clones having nucleotide sequence comprising the splice variants identified in Example 2 (hereafter referred to as RIPK2sv1.1, RIPK2sv1.2, or RIPK2sv2) are isolated using a 5′ “forward” RIPK2 primer and a 3′ “reverse” RIPK2 primer, to amplify and clone the entire RIPK2sv1.1, RIPK2sv1.2 or RIPK2sv2 mRNA coding sequences, respectively. The same 5′ “forward” primer is designed for isolation of full length clones corresponding to the RIPK2sv1.1 and RIPK2sv2 splice variants and has the nucleotide sequence of 5′ ATGAACGGGGAGGCCATCTGC AGCGCCC 3′ [SEQ ID NO 15]. The 5′ “forward” RIPK2sv1.2 primer is designed to have the nucleotide sequence of 5′ ATGACTCCTCCTTTACTTCATCATGACT 3′ [SEQ ID NO 16]. The same 3′ “reverse” primer is designed for isolation of full length clones corresponding to the RIPK2sv1.2 and RIPK2sv2 splice variants and has the nucleotide sequence of 5′ TTACATGCTT TTATTTTGAAGTAAATTT 3′ [SEQ ID NO 17]. The 3′ “reverse” RIPK2sv1.1 primer is designed to have the nucleotide sequence of 5′ TCAATGGCCAAGCAACATCAGGATATTC 3′ [SEQ ID NO 18].

RT-PCR

The RIPK2sv1.1, RIPK2sv1.2 and RIPK2sv2 cDNA sequences are cloned using a combination of reverse transcription (RT) and polymerase chain reaction (PCR). More specifically, about 25 ng of fetal brain polyA mRNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10×buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the RIPK2 “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted RIPK2sv1.1, RIPK2sv1.2 and RIPK2sv2 mRNAs, products of about 207, 1212 and 1533 bases, respectively. The remainder of the 50 μl PCR reactions from fetal brain is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC 110A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified RIPK2sv1.1, RIPK2sv1.2 and RIPK2sv2 RT-PCR products from fetal brain are used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2X LB medium. These liquid cultures are incubated overnight on a roller at 37° C. Plasmid DNA is extracted from these cultures using the Qiagen (Valencia, Calif.) Qiaquik Spin Miniprep kit. Twelve putative RIPK2sv1.1, RIPK2sv1.2 and RIPK2sv2 clones, respectively are identified and prepared for a PCR reaction to confirm the presence of the expected RIPK2sv1.1 exon 1 to exon 3 and RIPK2sv2 exon 7 to exon 9 splice variant structures. A 25 μl PCR reaction is performed as described above (RT-PCR section) to detect the presence of RIPK2sv1.1, except that the reaction includes miniprep DNA from the TOPO TA/RIPK2sv1.1 ligation as a template. An additional 25 μl PCR reaction is performed as described above (RT-PCR section) to detect the presence of RIPK2sv1.2, except that the reaction includes miniprep DNA from the TOPO TA/RIPK2sv1.2 ligation as a template. An additional 25 μl PCR reaction is performed as described above (RT-PCR section) to detect the presence of RIPK2sv2, except that the reaction includes miniprep DNA from the TOPO TA/RIPK2sv2 ligation as a template. About 10 μl of each 25 μl PCR reaction is run on a 1% Agarose gel and the DNA bands generated by the PCR reaction are visualized and photographed on a UV light box to determine which minipreps samples have PCR product of the size predicted for the corresponding RIPK2sv1.1, RIPK2sv1.2, and RIPK2sv2 splice variant mRNAs. Clones having the RIPK2sv1.1 structure are identified based upon amplification of an amplicon band of 207 base pairs, whereas a normal reference RIPK2 clone will give rise to an amplicon band of 361 base pairs. Clones having the RIPK2sv2 structure are identified based upon amplification of an amplicon band of 1533 base pairs, whereas a normal reference RIPK2 clone would give rise to an amplicon band of 1623 base pairs. DNA sequence analysis of the RIPK2sv1.1 or RIPK2sv2 cloned DNAs confirm a polynucleotide sequence representing the deletion of exon 2 in the case of RIPK2sv1.1 or the deletion of exon 8 in the case of RIPK2sv2. Both the normal reference RIPK2 and a clone having the RIPK2sv1.2 structure give rise to an amplicon of 1212 base pairs. DNA sequence analysis of the RIPK2sv1.2 cloned DNAs confirm a polynucleotide sequence representing SEQ ID NO 3.

The polynucleotide sequence of RIPK2sv1 mRNA contains two open reading frames that encode an amino-terminal and a carboxy-terminal protein, referred to herein as RIPK2sv1.1 and RIPK2sv1.2, respectively. SEQ ID NO 1 encodes the amino terminal RIPK2sv1.1 protein (SEQ ID NO 2) that is similar to the reference RIPK2 protein (NP_{—}003812), but lacking the amino acids encoded by a 154 base pair region corresponding to exon 2 of the full length coding sequence of reference RIPK2 mRNA (NM_{—}003821.2). Deletion of the 154 basepair region results in a protein translation reading frame that has a frame shift and a premature stop codon in comparison to the reference RIPK2 protein reading frame. Therefore the first 57 amino acids of the RIPK2sv1.1 protein are identical to the reference RIPK2 (NP_{—}003812), but the next 11 amino acids are unique to the RIPK2sv1.1 protein as compared to the reference RIPK2 (NP_{—}003812). RIPK2sv1.2 polynucleotide (SEQ ID NO 3) encodes the carboxy terminal RIPK2sv1.2 protein (SEQ ID NO 4). While the polynucleotide sequence of SEQ ID NO 3 is identical to the last 1,212 nucleotides of the RIPK2 reference sequence NM_{—}003821.2, the RIPK2sv1.2 protein is translated using a different reading frame as compared to the reference RIPK2 protein (NP_{—}003812). Thus, the 403 amino acid long RIPK2sv1.2 protein has a completely unique amino acid sequence as compared to the reference RIPK2 protein (NP_{—}003812).

The polynucleotide sequence of RIPK2sv2 mRNA (SEQ ID NO 5) contains an open reading frame that encodes a RIPK2sv2 protein (SEQ ID NO 6) similar to the reference RIPK2 protein (NP_{—}003812), but lacking amino acids encoded by exon 8 of the full length coding sequence of reference RIPK2 mRNA (NM_{—}003821.2). The alternative splicing of exon 7 to exon 9 deletes a 90 base pair region corresponding to exon 8, but the protein reading frame at the novel exon 7/exon 9 splice junction is maintained in the same reading frame as that used to encode the reference RIPK2 protein (NP_{—}003812). Therefore the RIPK2sv2 protein is missing an internal 30 amino acid region that corresponds to the amino acids encoded by exon 8 as compared to the reference RIPK2 (NP_{—}003812).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 5, or the complement thereof.

2. The purified nucleic acid of claim 1, wherein said nucleic acid comprises a region encoding SEQ ID NO 6.

3. The purified nucleic acid of claim 1, wherein said nucleotide sequence encodes a polypeptide consisting of SEQ ID NO 6.

4. A purified polypeptide comprising SEQ ID NO 6.

5. The polypeptide of claim 4, wherein said polypeptide consists of SEQ ID NO 6.

6. An expression vector comprising a nucleotide sequence encoding SEQ ID NO 6, wherein said nucleotide sequence is transcriptionally coupled to an exogenous promoter.

7. The expression vector of claim 6, wherein said nucleotide sequence encodes a polypeptide consisting of SEQ ID NO 6.

8. The expression vector of claim 6, wherein said nucleotide sequence comprises SEQ ID NO 5.

9. The expression vector of claim 6, wherein said nucleotide sequence consists of SEQ ID NO 5.

10. A method of screening for compounds that is able to bind selectively to RIPK2sv2 comprising the steps of:
(a) providing a RIPK2sv2 polypeptide comprising SEQ ID NO 6;
(b) providing one or more RIPK2 isoform polypeptides that are not RIPK2sv2;
(c) contacting said RIPK2sv2 polypeptide and said RIPK2 isoform polypeptide that is not RIPK2sv2 with a test preparation comprising one or more compounds; and
(d) determining the binding of said test preparation to said RIPK2sv2 polypeptide and to said RIPK2 isoform polypeptide that is not RIPK2sv2, wherein a test preparation that binds to said RIPK2sv2 polypeptide, but does not bind to said RIPK2 polypeptide that is not RIPK2sv2, contains a compound that selectively binds said RIPK2sv2 polypeptide.

11. The method of claim 10, wherein said RIPK2sv2 polypeptide is obtained by expression of said polypeptide from an expression vector comprising a polynucleotide encoding SEQ ID NO 6.

12. The method of claim 11, wherein said polypeptide consists of SEQ ID NO 6.

13. A method of screening for a compound able to bind to or interact with a RIPK2sv2 protein or a fragment thereof comprising the steps of:
(a) expressing a RIPK2sv2 polypeptide comprising SEQ ID NO 6 or fragment thereof from a recombinant nucleic acid;
(b) providing to said polypeptide a labeled RIPK2 ligand that binds to said polypeptide and a test preparation comprising one or more compounds; and
(c) measuring the effect of said test preparation on binding of said labeled RIPK2 ligand to said polypeptide, wherein a test preparation that alters the binding of said labeled RIPK2 ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said RIPK2sv2 ligand is an RIPK2 inhibitor.

18. The method of claim 16, wherein said expression vector comprises SEQ ID NO 5 or a fragment of SEQ ID NO 5.

19. The method of claim 13, wherein said polypeptide comprises SEQ ID NO 6 or a fragment of SEQ ID NO 6.

20. The method of claim 13, wherein said test preparation contains one compound.


SEQUENCE LISTING (18 sequences)

SEQ ID NO: 1 - DNA, 207 bp, Homo sapiens
atgaacggggaggccatctgcagcgccctgcccaccattccctaccacaaactcgccgacctgcgctacctgagccgcggcgcctctggcactgtgtcgtccgcccgccacgcagactggcgcgtccaggtggccgtgaagcacctgcacatccacactccgctgctcgacagaaaactgaatatcctgatgttgcttggccattga

SEQ ID NO: 2 - protein, 68 aa, Homo sapiens
MNGEAICSALPTIPYHKLADLRYLSRGASGTVSSARHADWRVQVAVKHLHIHTPLLDRKLNILMLLGH

SEQ ID NO: 3 - DNA, 1212 bp, Homo sapiens
atgactcctcctttacttcatcatgacttgaagactcagaatatcttattggacaatgaatttcatgttaagattgcagattttggtttatcaaagtggcgcatgatgtccctctcacagtcacgaagtagcaaatctgcaccagaaggagggacaattatctatatgccacctgaaaactatgaacctggacaaaaatcaagggccagtatcaagcacgatatatatagctatgcagttatcacatgggaagtgttatccagaaaacagccttttgaagatgtcaccaatcctttgcagataatgtatagtgtgtcacaaggacatcgacctgttattaatgaagaaagtttgccatatgatatacctcaccgagcacgtatgatctctctaatagaaagtggatgggcacaaaatccagatgaaagaccatctttcttaaaatgtttaatagaacttgaaccagttttgagaacatttgaagagataacttttcttgaagctgttattcagctaaagaaaacaaagttacagagtgtttcaagtgccattcacctatgtgacaagaagaaaatggaattatctctgaacatacctgtaaatcatggtccacaagaggaatcatgtggatcctctcagctccatgaaaatagtggttctcctgaaacttcaaggtccctgccagctcctcaagacaatgattttttatctagaaaagctcaagactgttattttatgaagctgcatcactgtcctggaaatcacagttgggacagcaccatttctggatctcaaagggctgcattctgtgatcacaagaccactccatgctcttcagcaataataaatccactctcaactgcaggaaactcagaacgtctgcagcctggtatagcccagcagtggatccagagcaaaagggaagacattgtgaaccaaatgacagaagcctgccttaaccagtcgctagatgcccttctgtccagggacttgatcatgaaagaggactatgaacttgttagtaccaagcctacaaggacctcaaaagtcagacaattactagacactactgacatccaaggagaagaatttgccaaagttatagtacaaaaattgaaagataacaaacaaatgggtcttcagccttacccggaaatacttgtggtttctagatcaccatctttaaatttacttcaaaataaaagcatgtaa

SEQ ID NO: 4 - protein, 403 aa, Homo sapiens
MTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKLQSVSSAIHLCDKKKMELSLNIPVNHGPQEESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 5 - DNA, 1533 bp, Homo sapiens
atgaacggggaggccatctgcagcgccctgcccaccattccctaccacaaactcgccgacctgcgctacctgagccgcggcgcctctggcactgtgtcgtccgcccgccacgcagactggcgcgtccaggtggccgtgaagcacctgcacatccacactccgctgctcgacagtgaaagaaaggatgtcttaagagaagctgaaattttacacaaagctagatttagttacattcttccaattttgggaatttgcaatgagcctgaatttttgggaatagttactgaatacatgccaaatggatcattaaatgaactcctacataggaaaactgaatatcctgatgttgcttggccattgagatttcgcatcctgcatgaaattgcccttggtgtaaattacctgcacaatatgactcctcctttacttcatcatgacttgaagactcagaatatcttattggacaatgaatttcatgttaagattgcagattttggtttatcaaagtggcgcatgatgtccctctcacagtcacgaagtagcaaatctgcaccagaaggagggacaattatctatatgccacctgaaaactatgaacctggacaaaaatcaagggccagtatcaagcacgatatatatagctatgcagttatcacatgggaagtgttatccagaaaacagccttttgaagatgtcaccaatcctttgcagataatgtatagtgtgtcacaaggacatcgacctgttattaatgaagaaagtttgccatatgatatacctcaccgagcacgtatgatctctctaatagaaagtggatgggcacaaaatccagatgaaagaccatctttcttaaaatgtttaatagaacttgaaccagttttgagaacatttgaagagataacttttcttgaagctgttattcagctaaagaaaacaaaggaatcatgtggatcctctcagctccatgaaaatagtggttctcctgaaacttcaaggtccctgccagctcctcaagacaatgattttttatctagaaaagctcaagactgttattttatgaagctgcatcactgtcctggaaatcacagttgggacagcaccatttctggatctcaaagggctgcattctgtgatcacaagaccactccatgctcttcagcaataataaatccactctcaactgcaggaaactcagaacgtctgcagcctggtatagcccagcagtggatccagagcaaaagggaagacattgtgaaccaaatgacagaagcctgccttaaccagtcgctagatgcccttctgtccagggacttgatcatgaaagaggactatgaacttgttagtaccaagcctacaaggacctcaaaagtcagacaattactagacactactgacatccaaggagaagaatttgccaaagttatagtacaaaaattgaaagataacaaacaaatgggtcttcagccttacccggaaatacttgtggtttctagatcaccatctttaaatttacttcaaaataaaagcatgtaa

SEQ ID NO: 6 - protein, 510 aa, Homo sapiens
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 7 - DNA, 20 bp, Homo sapiens
tgctcgacagaaaactgaat

SEQ ID NO: 8 - DNA, 20 bp, Homo sapiens
gaaaacaaaggaatcatgtg

SEQ ID NO: 9 - protein, 10 aa, Homo sapiens
TPLLDRKLNI

SEQ ID NO: 10 - protein, 10 aa, Homo sapiens
LKKTKESCGS

SEQ ID NO: 11 - DNA, 26 bp, Homo sapiens
ctgcccaccattccctaccacaaact

SEQ ID NO: 12 - DNA, 28 bp, Homo sapiens
cgccactttgataaaccaaaatctgcaa

SEQ ID NO: 13 - DNA, 28 bp, Homo sapiens
atatcctgatgttgcttggccattgaga

SEQ ID NO: 14 - DNA, 28 bp, Homo sapiens
tcatggagctgagaggatccacatgatt

SEQ ID NO: 15 - DNA, 28 bp, Homo sapiens
atgaacggggaggccatctgcagcgccc

SEQ ID NO: 16 - DNA, 28 bp, Homo sapiens
atgactcctcctttacttcatcatgact

SEQ ID NO: 17 - DNA, 28 bp, Homo sapiens
ttacatgcttttattttgaagtaaattt

SEQ ID NO: 18 - DNA, 28 bp, Homo sapiens
tcaatggccaagcaacatcaggatattc